(19) United States
(12) Patent Application Publication
(10) Pub. No.: US 2009/0163806 A1
(43) Pub. Date: Jun. 25, 2009

(54) ULTRASONIC DIAGNOSTIC APPARATUS AND ULTRASONIC STRESS IMAGE ACQUISITION METHOD

(75) Inventors: Kazutoshi Sadamitsu, Otawara-shi (JP); Yasuhiko Abe, Otawara-shi (JP); Osamu Nakajima, Otawara-shi (JP); Yutaka Kobayashi, Nasushiobara-shi (JP); Yoko Okamura, Nasushiobara-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-Shi (JP)

(21) Appl. No.: 12/335,996

(22) Filed: Dec. 16, 2008

(30) Foreign Application Priority Data
Dec. 20, 2007 (JP) 2007-328919

(51) Int. Cl. A61B 8/00

(52) U.S. Cl. 600/438

(57) ABSTRACT

An ultrasonic diagnostic apparatus comprises a condition defining/stress data acquiring section for defining a predetermined strain processing condition, radiating an ultrasonic wave to a tissue of a subject to be examined and acquiring stress image data according to the reception signal obtained from the reflected ultrasonic wave in the state of the tissue before bearing a load put thereon, a processing condition storing/defining section for storing the strain processing conditions, an automatically defining/stress data acquiring section for automatically defining the strain processing condition stored in the processing condition storing/defining section and acquiring stress image data on the tissue in a loaded state of the tissue after bearing a load put thereon, a tissue strain data acquiring section for executing a tissue strain imaging process on the stress image data and acquiring tissue strain image data and an image display section for displaying a stress image according to the tissue strain data.

### CROSS REFERENCE TO RELATED APPLICATION

This invention is based upon and claims the benefit of priority from the prior Japanese Patent Application No. 2007-328919, filed on Dec. 20, 2007, the entire contents of which are incorporated herein by reference.

### BACKGROUND OF THE INVENTION

(1) Field of the Invention

This invention relates to an ultrasonic diagnostic apparatus and an ultrasonic image display apparatus. More particularly, the present invention relates to an ultrasonic diagnostic apparatus and an ultrasonic stress image acquisition method to be used for processing the strain of a heart or some other organ according to the ultrasonic reception signal acquired from a subject of examination.

(2) Description of the Related Art

An ultrasonic diagnostic apparatus is for acquiring an image of the interior of the body of a subject to be physically examined by irradiating an ultrasonic wave onto the subject from an ultrasonic oscillator and receiving and electrically processing the reflected wave. Such an apparatus is operated typically for diagnosing a living organ in terms of profile and function.

Stress echo techniques of putting a load of exercise or drug on a subject to be physically examined, collecting ultrasonic image data of the subject in the loaded state by exercise or drug and evaluating the mobility of the myocardium are being popularly employed for diagnosing the function of the heart of a subject (refer to, e.g., Jpn. Pat. Appln. Laid-Open Publication No. 2007-135994).

When a stress echo technique is employed for examining the heart of a subject, data on the heart need to be collected for a plurality of times from so many predetermined positions on predefined conditions. The image data acquired from each of the positions are referred to as a view (of the shot site). If a load of exercise is put on the heart of the subject, the heart is checked for its condition when the heart is at rest before the exercise, or in a pre-exercise phase, when the heart is bearing the load of the exercise, or in an intra-exercise phase, and when the heart is at rest once again after the exercise, or in a post-exercise phase. In a stress echo examination, data on each view are acquired in each phase. Conventionally, the conditions on which a predetermined view is acquired (strain processing conditions) in a phase are redefined each time after acquiring a view.

On the other hand, tissue strain imaging (TSI) techniques of mapping and displaying pieces of information acquired on the contraction in the direction of the long axis and on the stretch in the direction of the thickness of the heart during a cardiac contraction phase are being employed (refer to, e.g., Jpn. Pat. Appln. Laid-Open Publication No. 2007-044499). Image processing using a TSI technique is normally conducted after acquiring data from the subject. While there is a temporal margin from the time when data are acquired to the time when a TSI image is obtained, a strain processing image needs to be obtained within a short period of time when a real time TSI technique is employed for obtaining an image on the spot.

However, there arises a problem that it takes a relatively long time to obtain TSI image data when strain processing conditions are redefined each time after acquiring a view and more particularly when a TSI image needs to be obtained on a real time basis.

### BRIEF SUMMARY OF THE INVENTION

In view of the above-identified problem that strain processing conditions needs to be redefined each time when acquiring strain image data so that it takes a long time before obtaining a strain image, it is therefore the object of the present invention to provide an ultrasonic diagnostic apparatus and an ultrasonic stress image acquisition method that can quickly acquire a stress image subjected to a TSI imaging process (tissue strain imaging process).

In an aspect of the present invention, the above object is achieved by providing an ultrasonic diagnostic apparatus including: a condition defining/stress data acquiring section for defining predetermined strain processing conditions, radiating an ultrasonic wave to a tissue of a subject to be examined and acquiring stress image data according to the reception signal obtained from the reflected ultrasonic wave in the state of the tissue before bearing a load put thereon; a processing condition storing/defining section for storing the strain processing condition; an automatically defining/stress data acquiring section for automatically defining the strain processing condition stored in the processing condition storing/defining section and acquiring stress image data on the tissue in a loaded state of the tissue after bearing a load put thereon; a tissue strain data acquiring section for executing a tissue strain imaging process on the stress image data and acquiring tissue strain image data; and an image display section for displaying a stress image according to the tissue strain data.

Thus, according to the present invention, there are provided an ultrasonic diagnostic apparatus and an ultrasonic stress image acquisition method that can quickly acquire a stress image subjected to a TSI image process (tissue strain imaging process).

### BRIEF DESCRIPTION OF THE DRAWINGS

FIG. 1 is a schematic block diagram according to embodiments of the present invention, illustrating the configuration thereof;

FIG. 2 is a flowchart of the operation of each of the embodiments of the present invention;

FIG. 3 is a schematic illustration of the operation of storing and automatically defining strain processing conditions according to the first embodiment of the present invention;

FIG. 4 is a schematic illustration of the operation of storing and automatically defining strain processing conditions according to a second embodiment of the present invention; and

FIGS. 5A through 5D are four exemplar views. FIG. 5A is a schematic cross sectional view of a heart taken along the long axis thereof. FIG. 5B is a schematic cross sectional view of a heart taken along the short axis thereof. FIG. 5C is a schematic two-chamber view of a heart. FIG. 5D is a schematic four-chamber view of a heart.

### DETAILED DESCRIPTION OF THE INVENTION

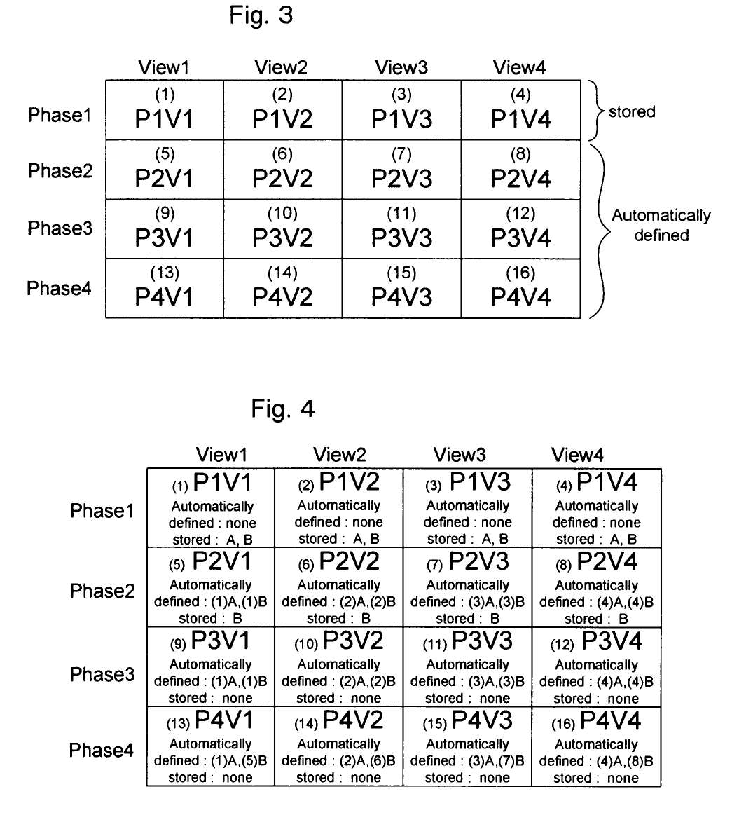

Now, the present invention will be described in greater detail by referring to the accompanying drawings that schematically illustrate preferred embodiments of the invention. Firstly, a case where four views of different sites of a tissue to be examined are obtained by shooting them in four phases before and after a load of exercise is put on the tissue will be described. In this case, four views, or Views 1 through 4, are obtained in each of the four phases, or Phases 1 through 4 as shown in FIG. 3. Note that each of the views in one of the phases is represented by PiVj (i=1 through 4, j=1 through 4). Therefore, View 1 in Phase 1 is expressed by P1V1. The strain processing conditions for obtaining each view typically include an angle, the pitch between two strain points and a color map of each strain.

When, for example, a definition of an angle is a strain processing condition, it means that the views are obtained from different respective angles. While an angle may be automatically defined for each view as a strain processing condition, the operator can correct the angle.

When an ultrasonic diagnostic apparatus is operated by means of a stress echo method, predetermined strain processing conditions are defined and an ultrasonic wave is transmitted. Then, stress image data are obtained from the reflected ultrasonic wave and processed by way of a TSI image process (tissue strain image process) to obtain tissue strain image data. Thus, a stress image is obtained for each view.

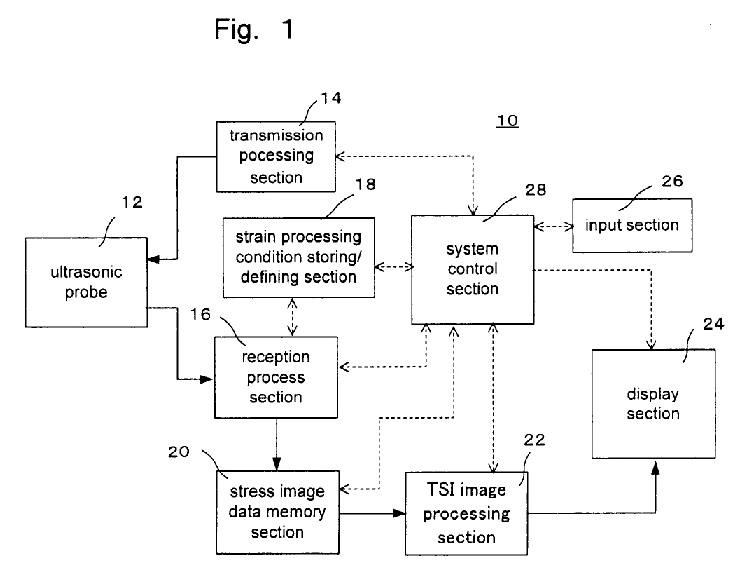

FIG. 1 is a schematic block diagram of the first and second embodiments of the present invention, illustrating the configuration thereof. As shown in FIG. 1, the ultrasonic diagnostic apparatus 10 has an ultrasonic probe 12 containing a plurality of ultrasonic wave oscillators (not shown) for transmitting and receiving an ultrasonic wave, a transmission processing section 14 for supplying a drive signal to the ultrasonic probe 12 for transmitting an ultrasonic wave, a reception process section 16 for executing a reception process on the ultrasonic wave received by the ultrasonic probe section 12, a strain processing conditions storing/defining section 18 for defining the strain processing conditions at the time of an ultrasonic shooting, a stress image data memory section 20 for acquiring data on each stress image from the reflected ultrasonic signal obtained by the reception processing section 16, a TSI image processing section 22 for executing a TSI image process (tissue strain imaging process) by using the stress image data stored in the stress image data memory section 20, a display section 24 for displaying the image obtained by the TSI image processing section 22 as a result of an image process, an input section 26 to be operated by an operator to input, for example, a program for automatically defining strain processing conditions, which will be described in greater detail hereinafter and a system control section 28 for controlling the operations of these component sections.

### First Embodiment

The first embodiment automatically defines strain processing conditions input for each of the views in Phase 1 when corresponding views are obtained in each of Phases 1 through 4. The strain processing conditions of this embodiment include an angle, the pitch between two strain points and a color map.

As an angle is defined, the center for correcting the angle for a short axis image of a heart, for instance, is defined. The pitch between two strain points for a short axis image differs from the pitch between two strain points for a short axis image.

The expression that a color map is a strain condition means that the moving distance of each site showing an upper limit value or a lower limit value of strain values 0 to +60 or −20 to 0 for each view in a same phase is indicated by a color. With such an arrangement, how each site bears a load can be recognized easily. The range to be indicated by a same color is unequivocally defined.

Now, the operation of the embodiment of ultrasonic diagnostic apparatus will be described below by referring to the drawings particularly in terms of the operation of defining strain processing conditions when four views are obtained in each of the phases and a tissue (the heart) of the subject is made to bear a load of exercise.

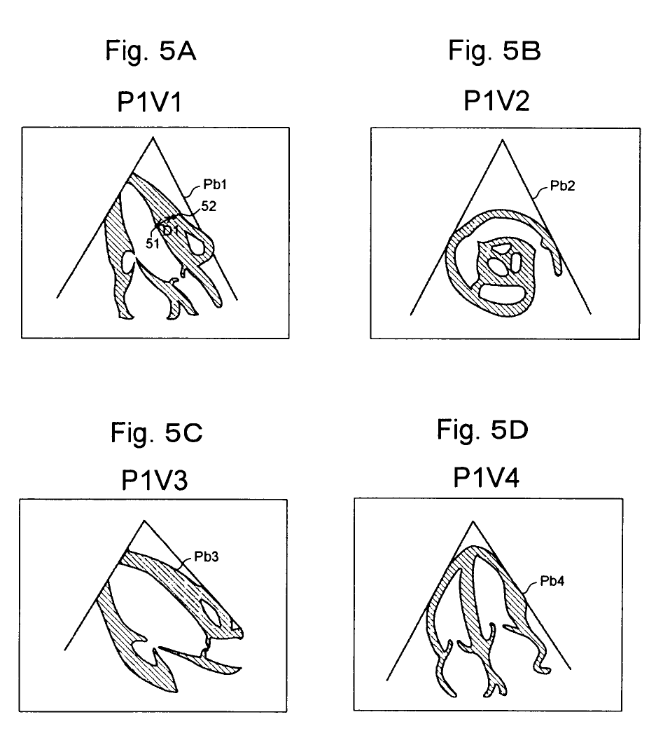

Firstly, the four views of the heart will be described specifically. For example, View 1 is a cross sectional view of the heart taken along the long axis of the heart as shown in FIG. 5A and View 2 is a cross sectional view of the heart taken along the short axis of the heart as shown in FIG. 5B. View 3 is a 2-chamber cross sectional view of the heart as shown in FIG. 5C and View 4 is a 4-chamber cross sectional view of the heart as shown in FIG. 5D. Pb1 through Pb4 indicate the images of the heart in cross section obtained by means of an ultrasonic probe.

If the strain processing conditions include the pitch between two strain points, the pitch refers to the distance D1 between Point 51 and Point 52 in FIG. 5A. If FIG. 5A is a view before a load of strain is put on the heart, it corresponds to stress image data P1V1 of View 1 in Phase 1. The distance D1 varies in the substance phases, or Phase 2 through Phase 4, including a phase in which a load of strain is not put on the heart yet, a phase in which the load of strain is being put on the heart and a phase in which the load of strain is moved away.

Referring now to FIG. 3, P1V1 indicates the stress image data of View 1 in Phase 1 and (1) preceding it shows that it is obtained first. Similarly, (2) through (16) respectively show the image data obtained second through sixteenth.

The strain processing conditions vary from view to view, although they remain substantially same in each phase. More specifically, the strain processing conditions of the stress image data P2V1, those of the stress image data P3V1 and those of the stress image data P4V1 are substantially same as those of the stress image data P1V1. So are the strain processing conditions of the stress image data in each of the other phases. Phases 1 through 4 typically refer to a phase immediately before a load of strain is put on the heart yet (and the heart is at rest), a phase in which the load of strain is being put on the heart, a phase in which the load of strain is moved away and a phase that comes when a predetermined period of time has passed since the time when the load of strain was moved away.

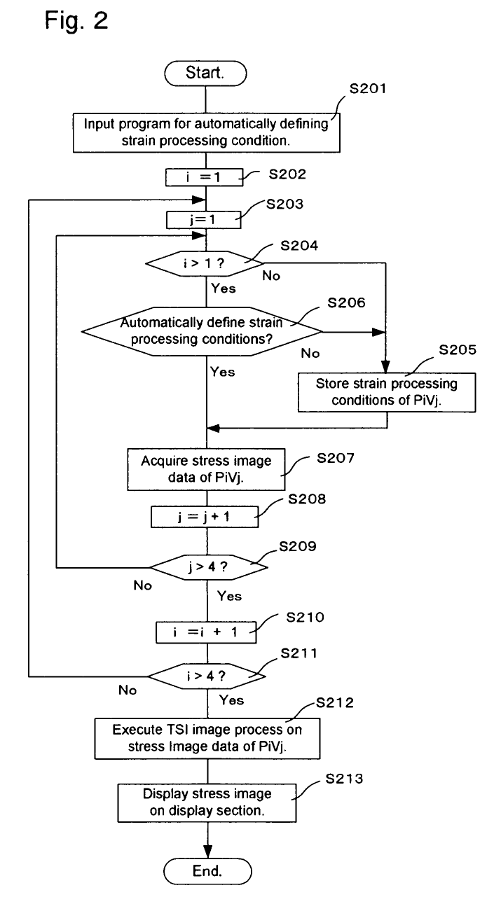

Now, referring to the flowchart of FIG. 2, the operator inputs a program for automatically defining strain processing conditions in Step S201. This processing operation is carried out as the operator inputs the program by means of a mouse or a keyboard in the input section 26. The control signal for the processing operation is input and stored in the strain processing conditions storing/defining section 18 by way of the system control section 28. The strain processing conditions typically include an angle, the pitch between two strain points and a color map of the strain.

While the strain processing conditions remain substantially same for a same view as pointed out above, the operator is prompted to input a command from the input section 26 for automatically defining the strain processing conditions obtained for View 1 in Phase 1 also for Phase 2, Phase 3 and Phase 4 or not. Then, the specified program is stored in the strain processing conditions storing/defining section 18. Assume here that the strain processing conditions for P1V1 through P1V4 are also automatically input and defined for P2V1 through P2V4, P3V1 through P3V4 and P4V1 through P4V4 in the following description of this embodiment.

When acquiring data for each view in Phase 1, the strain processing conditions used for the immediately preceding image are stored in the strain processing conditions storing/defining section 18 as effective conditions.

More specifically, Phase 1 is specified as i=1 in Step S202 and if View 1 is specified as j=1 in Step S203. Then, it is checked in Step S203 if i is greater than 1 or not. The process proceeds to Step S205 to store the strain processing conditions on which the stress image data for P1V1 are acquired in the strain processing conditions storing/defining section 18 because i=1.

Then, in Step S207, the strain image data for P1V1 are acquired and stored in the stress image data memory section 20.

Then, in Step S208, j=i+1 is defined and, in Step S209, it is checked if j becomes greater than 4 or not. If j is not greater than 4, the process proceeds to Step S204, where it is checked if i is greater than 1 or not. As long as i is not greater than 1, the strain processing conditions for acquiring stress image data are stored in the strain processing conditions storing/defining section 18 for each of Views 1 through 4 in Phase 1 (P1V1 through P1V4 in FIG. 3) in Step S205. Then, the stress image data of P1V1 through P1V4 are acquired in Step S207.

If it is determined in Step S209 that j is greater than 4, the process moves to Step S210, where i is turned to i+1. In the next step, or Step S211, it is checked if i is greater than 4 or not. If i is equal to 2, the process returns from Step S211 to Step S203, where j=1 is defined. In the next step, or Step S204, it is checked if i is greater than 1 or not. Since i is greater than 1 now, the process moves to Step S206, where it is determined if strain processing conditions are automatically defined for the current i according to the strain processing conditions stored in Phase 1 or not.

If it is determined in Step S206 that strain processing conditions are automatically defined according to the strain processing conditions stored in Phase 1, they are then automatically defined and stress image data are acquired in Step S207.

If, on the other hand, it is determined in Step S206 that strain processing conditions are not automatically defined, the process returns to Step S205, where the current strain processing conditions are automatically stored, and then stress image data are acquired in Step S207.

Data for Views 1 through 4, or stress image data for P2V1 through P2V4 shown in FIG. 3, are acquired in Phase 2. The strain processing conditions in Phase 1 are automatically defined as strain processing conditions for each of the views in Phase 1. Therefore, strain processing conditions are automatically defined in Step S206 and the process proceeds to the next step, or Step S207. The process never returns from Step S206 to Step S205 to store the strain processing conditions.

In this way, stress image data are acquired for each of the views in Phase 2 and the process proceeds from Step S209 to Step S210, where i is turned to 3. Then, the process returns from the next step, or Step S211, to Step S203 because i is now equal to 3. In Phase 3, the strain processing conditions of the views in Phase 1 are automatically defined as in Phase 2. In other words, the process proceeds from Step S206 to Step S207, where stress image data that correspond to all the views in Phase 3 (P3V1 through P3V4) are acquired.

As the data for all the views in Phase 3 are acquired, i is turned to 4 in Step S210 and the process returns from the next step, or Step S211, to Step S203 once again.

In Phase 4, the process proceeds through Steps S204, S206, S207, S208 and S209. Strain processing conditions are automatically defined in Step S206 as in Phase 1 and view data (stress image data) for P4V1, P4V2, P4V3 and P4V4 are acquired in Step S207.

In this way, stress image data P1V1 through P1V4, P2V1 through P2V4, P3V1 through P3V4 and P4V1 through P4V4 that correspond to the respective views listed in FIG. 3 are acquired and stored in the stress image data memory section 20.

In the next step, of Step S212, tissue strain image data are acquired from the sixteen stress image data by the TSI image processing section 22 shown in FIG. 1 by way of the TSI process. The tissue strain image data are then sent to the display section 24, which displays stress images on the display screen according to the tissue strain image data.

Thus, with this embodiment, the heart is at rest in Phase 1 so that the operator can take time for defining conditions.

### Second Embodiment

Now, the second embodiment will be described below by referring to FIG. 4 and also to the flowchart of FIG. 2. In FIG. 4, (1) through (16) indicates the order in which the view data (the stress image data) of the views are acquired.

The strain processing conditions of this embodiment typically include A (angle) and B (color map). While A (angle) is automatically defined just like B, it needs to be corrected sometime later.

The process of this embodiment also proceeds basically according to the flowchart shown in FIG. 2. However, when there are two or more than two strain processing conditions, each of them is determined in Steps S206, S205 and S207.

The operator inputs a program for automatically defining strain processing conditions from the input section 26 in Step S201 shown in FIG. 2. The program for automatically defining strain processing conditions is then stored in the strain processing conditions storing/defining section 18 by way of the system control section 28. Assume here that the strain processing conditions as shown in FIG. 4 are input for this embodiment. In FIG. 4, the numeral in parentheses that is shown immediately before each of the strain processing conditions (A) and (B) indicates the order in which the condition is acquired. For example, the strain processing condition (1) A for P2V1 is the strain processing condition for (1) (P1V1).

Thus, strain processing conditions (A: angle, B: color map) of each of the views are stored in Phase 1. In Phase 2, the strain processing conditions of each of the views in the immediately preceding phase, or Phase 1, are automatically defined for the corresponding view in Phase 2 and the strain processing condition B is stored. For example, A (angle) and B (color map) of View 1 in Phase 1 are automatically defined as the strain processing conditions of P2V1 and the strain processing condition B that is used for acquiring the P2V1 data is stored.

In Phase 3, the strain processing conditions of each of the views in Phase 1 are automatically defined for the corresponding view in Phase 3 but the strain processing conditions for the corresponding view in Phase 3 are not stored. For example, only A (angle) and B (color map) of View 1 in Phase 1 are automatically defined as the strain processing conditions of P3V1 but the strain processing conditions that are used for acquiring P3V1 data are not stored.

In Phase 4, the strain processing condition A of each of the views in Phase 1 and the strain processing condition B of each of the views in Phase 2 are automatically defined for the corresponding view in Phase 3 but the strain processing conditions for the corresponding view in Phase 4 are not stored. For example, A of View 1 in Phase 1 and B of View 1 in Phase 2 are automatically defined as the strain processing conditions of P4V1.

Now, the processing operations after Step S202 in FIG. 2 will be described below. In Step S202, i=1 is defined and Phase 1 is specified. In Step S203, j=1 is defined and P1V1 is specified. Then, the process proceeds from Step S204 to Step S205, where the strain processing conditions A and B of P1V1 are stored and, in Step S207, stress image data are acquired for the view and stored in the stress image data memory section 20. Similarly, the strain processing conditions A and B are stored for Phase 1 in Step S205 and stress image data for Views 1 through 4 are acquired in Phase 1 and stored in the stress image data memory section 20.

If it is determined in Step S209 that i is equal to 1 (Phase 1) and j is greater than 4, i is turned to 2 in Step S210 and the process returns from Step S211 to Step S203 and then proceeds to the processing operations in Phase 2. The process moves from Step S204 to Step S206 and the strain processing conditions A and B for the corresponding view in Phase 1 are automatically defined for the current view in Phase 2. Although not shown in FIG. 2, the strain processing condition B for acquiring the view in Phase 2 is stored.

Subsequently, stress image data for Views P2V1 through P2V4 are acquired in Step S207.

In Phase 3, the strain processing conditions A and B of the corresponding view are automatically defined as the strain processing conditions A and B of the current view. However, the strain processing conditions A and B of the current view in Phase 3 are not stored. Stress image data of each of the views (P3V1 through P3V4) in Phase 3 are acquired in Step S207.

As i is turned to 4 in Step S210 and the process returns from Step S211 to Step S203, j is defined as 1 in Step S203 and the strain processing conditions corresponding to P4V1 are defined and stress image data are acquired for the view.

In Phase 4, the strain processing condition A in Phase 1 is also defined as the strain processing condition A in Phase 4 but the strain processing condition B in Phase 2 is defined as the strain processing condition B in Phase 4. The strain processing conditions A and B of the current view in Phase 4 are not stored.

For example, if i=4 and j=1, the process moves from Step S204 to Step S206, where it is determined that strain processing conditions are to be automatically defined or not. (1) A and (5) B are automatically defined for P4V1 as shown in FIG. 4. In other words, the strain processing condition A for View 1 in Phase 1 (P1V1) and strain processing condition B for View 1 in Phase 2 (P2V1) are automatically defined as the strain processing conditions for P4V1. However, the strain processing conditions for P4V1 are not stored.

In Phase 4, strain processing conditions are automatically defined for each of the views in Step S206 and stress image data are acquired in Step S207 and stored in the stress image data memory section 20 for the view. After the stress image data of P4V1 through P4V4 are acquired and stored, the process moves to Step S212, where the TSI image processing section 22 executes a TSI image process on those data and the corresponding images are displayed on the display section 24 in Step S213.

Note that the strain processing condition A (angle) of each of the views in Phase 1 is automatically defined as the strain processing condition of each of the views in Phase 4 while the strain processing condition B (color map) of each of the views in Phase 2 is defined as the strain processing condition of each of the views in Phase 4.

With this embodiment, Phase 2 and on are those in which the tissue bears a load and the diagnosis needs to be given within a limited period of time. However, the time necessary for the definitions can be made very short to a great advantage for a diagnosis.

While the tissue to be examined is the heart of the subject in the above description of the embodiments, the present invention can be applied to any other tissue. While the embodiments are described above in terms of an instance of putting a load of exercise on the heart of the subject of physical examination, the present invention can also be applied to instance of putting a load of drug of the heart of the subject of physical examination.

While the strain processing conditions include an angle, the pitch between two strain points and a color map or an angle or a color map in each of the above-described embodiments, strain processing conditions are by no means limited thereto.

While stress image data are acquired in four phases and four views are acquired in each of the phases in the above-described embodiments, the present invention is by no means limited thereto and some other number of phases and/or some other number of views may be selected for the purpose of the present invention.

The present invention is by no means limited to the above-described embodiments particularly in terms of storing strain processing conditions, automatically defining strain processing conditions and selecting parameters and some other arrangement may be appropriately selected for the purpose of the present invention.

Obviously, many modifications and variations of this invention are possible in the light of the above teachings. It is therefore to be understood that within the scope of the appended claims, this invention may be practiced otherwise than as specification.

What is claimed is:

1. An ultrasonic diagnostic apparatus comprising:
a condition defining/stress data acquiring section for defining predetermined a strain processing condition, radiating an ultrasonic wave to a tissue of a subject to be examined and acquiring stress image data according to the reception signal obtained from the reflected ultrasonic wave in the state of the tissue before bearing a load put thereon;
a processing condition storing/defining section for storing the strain processing conditions;
an automatically defining/stress data acquiring section for automatically defining the strain processing condition stored in the processing condition storing/defining section and acquiring stress image data on the tissue in a loaded state of the tissue after bearing a load put thereon;
a tissue strain data acquiring section for executing a tissue strain imaging process on the stress image data and acquiring tissue strain image data; and
an image display section for displaying a stress image according to the tissue strain data.

2. The apparatus according to claim 1, wherein
the strain processing condition is the pitch between two strain points, an angle, or a color map.

3. The apparatus according to claim 2, wherein
the loaded state of the tissue after bearing a load put thereon is the state of the tissue that is observed immediately after removing the load put on the tissue and when a predetermined period of time passes after removing the load.

4. The apparatus according to claim 3, wherein
the tissue is the heart of the subject and the plurality of views are a long axis cross sectional view of the heart, a short axis cross sectional view of the heart, a 2-chamber cross sectional view and a 4-chamber cross sectional view.

5. The apparatus according to claim 1, wherein
the strain processing condition includes an angle and a color map.

6. The apparatus according to claim 5, wherein
the loaded state of the tissue after bearing a load put thereon is the state of the tissue that is observed immediately after removing the load put on the tissue and when a predetermined period of time passes after removing the load.

7. The apparatus according to claim 6, wherein
the tissue is the heart of the subject and the plurality of views are a long axis cross sectional view of the heart, a short axis cross sectional view of the heart, a 2-chamber cross sectional view and a 4-chamber cross sectional view.

8. An ultrasonic diagnostic apparatus comprising:
a condition defining/stress data acquiring section for acquiring stress image data according to the reception signal obtained from the reflected wave of an irradiated ultrasonic wave in order to acquire different views of a tissue of a subject of physical examination by defining a predetermined strain processing condition in the first phase of the state of the tissue before putting on a load on the tissue;
a processing condition storing/defining section for storing the strain processing condition for each of the different views in the first phase;
an automatically defining/stress data acquiring section for automatically defining the strain processing condition stored in the first phase in the processing condition storing/defining section as the strain processing condition for the view corresponding to the view in the first phase in each of the second and subsequent phases and acquiring stress image data on the state of the tissue after putting a load on the tissue;
a tissue strain data acquiring section for executing a tissue strain imaging process on the stress image data and acquiring tissue strain image data; and
an image display section for displaying a stress image according to the tissue strain image data.

9. The apparatus according to claim 8, wherein
the strain processing condition stored in the processing condition storing/defining section includes a plurality of conditions including a color map and the processing condition storing/defining section automatically defines strain processing conditions for the view corresponding to the first phase as strain processing conditions for the views in the second and subsequently phases and stores the strain processing condition of the color map of the view in the second phase.

10. The apparatus according to claim 8, wherein
the strain processing condition is the pitch between two strain points, an angle, or a color map.

11. The apparatus according to claim 10, wherein
the second phase comes immediately after removing the load put on the tissue and each of the subsequent phases comes when a predetermined period of time passes after removing the load put on the tissue.

12. The apparatus according to claim 11, wherein
the tissue is the heart of the subject and the plurality of views are a long axis cross sectional view of the heart, a short axis cross sectional view of the heart, a 2-chamber cross sectional view and a 4-chamber cross sectional view.

13. The apparatus according to claim 8, wherein
the strain processing condition includes an angle and a color map.

14. The apparatus according to claim 13, wherein
the second phase comes immediately after removing the load put on the tissue and each of the subsequent phases comes when a predetermined period of time passes after removing the load put on the tissue.

15. The apparatus according to claim 14, wherein
the tissue is the heart of the subject and the plurality of views are a long axis cross sectional view of the heart, a short axis cross sectional view of the heart, a 2-chamber cross sectional view and a 4-chamber cross sectional view.

16. An ultrasonic stress image acquisition method comprising:
a condition defining/stress data acquiring step of acquiring stress image data according to the reception signal obtained from the reflected wave of an irradiated ultrasonic wave in order to acquire different views of a tissue of a subject of physical examination by defining a predetermined strain processing condition in the first phase of the state of the tissue before putting on a load on the tissue;
a processing condition storing step of storing the strain processing condition for each of the different views in the first phase;
an automatically defining/stress data acquiring step of automatically defining the strain processing condition stored in the first phase in the processing condition storing step as the strain processing condition for the view corresponding to the view in the first phase in each of the second and subsequent phases and acquiring stress image data on the state of the tissue after putting a load on the tissue; and
a tissue strain data acquiring step of executing a tissue strain imaging process on the stress image data and acquiring tissue strain image data.

17. The method according to claim 16, wherein
the strain processing condition is the pitch between two strain points, an angle, or a color map.

18. The method according to claim 17, wherein
the second phase comes immediately after removing the load put on the tissue and each of the subsequent phases comes when a predetermined period of time passes after removing the load put on the tissue.

19. The method according to claim 18, wherein
the tissue is the heart of the subject and the plurality of views are a long axis cross sectional view of the heart, a short axis cross sectional view of the heart, a 2-chamber cross sectional view and a 4-chamber cross sectional view.

20. The method according to claim 16, wherein
the strain processing condition includes an angle and a color map.

21. The method according to claim 20, wherein
the second phase comes immediately after removing the load put on the tissue and each of the subsequent phases comes when a predetermined period of time passes after removing the load put on the tissue.

22. The method according to claim 21, wherein
the tissue is the heart of the subject and the plurality of views are a long axis cross sectional view of the heart, a short axis cross sectional view of the heart, a 2-chamber cross sectional view and a 4-chamber cross sectional view.
